(19) United States
(12) Patent Application Publication
(10) Pub. No.: US 2021/0403414 A1
(43) Pub. Date: Dec. 30, 2021

(54) PRODUCTION OF NITROGEN-CONTAINING CHELATORS

(71) Applicant: Ascend Performance Materials Operations LLC, Houston, TX (US)

(72) Inventors: Huayun Yu, Houston, TX (US); Zhensheng Ding, Houston, TX (US); Nick Sparkman, Houston, TX (US); Thomas Peavy, Houston, TX (US); Ernst Bezemer, Houston, TX (US); Kyle Kissell, Houston, TX (US)

(73) Assignee: Ascend Performance Materials Operations LLC, Houston, TX (US)

(21) Appl. No.: 17/363,841

(22) Filed: Jun. 30, 2021

Related U.S. Application Data
(60) Provisional application No. 63/046,213, filed on Jun. 30, 2020.

(51) Int. Cl. C07C 227/26; C07C 253/10; C07C 253/30

(52) U.S. Cl. CPC C07C 227/26; C07C 253/30; C07C 253/10

(57) ABSTRACT

Reaction pathways and conditions for the production of nitrogen-containing chelators, such as a glycine derivative, are described herein. In particular, the present disclosure describes a process for the production of a nitrile intermediate by reacting a tetra-amino compound with an aldehyde and a hydrogen cyanide to form the nitrile intermediate. The nitrile intermediate may then be further processed to produce the chelators at a high yield and/or a high purity.

### CROSS REFERENCE TO RELATED APPLICATIONS

This application claims priority to U.S. Provisional Application No. 63/046,213, filed Jun. 30, 2020, which is incorporated herein by reference.

### FIELD

The present disclosure relates generally to the production of nitrogen-containing chelators. In particular, the present disclosure relates to reaction pathways and conditions for the production of nitrogen-containing chelators with high yield and/or purity.

### BACKGROUND

Chelators, also known as chelating agents, are organic compounds whose structures allow them to form bonds to a metal atom. Because chelators typically form two or more separate coordinate bonds to a single, central metal atom, chelators can be described as polydentate ligands. Chelators often include sulfur, nitrogen, and/or oxygen, which act as electron-donating atoms in bonds with the metal atom.

Chelators are useful in a variety of applications, where their propensity to form chelate complexes with metal atoms is important. Conventional uses of chelators include in nutritional supplements, in medical treatments (e.g., chelation therapy to remove toxic metals from the body), as contrast agents (e.g., in MRI scans), in domestic and/or industrial cleaners and/or detergents, in the manufacture of catalysts, in removal of metals during water treatment, and in fertilizers. For example, chelators play an important role in treatment of cadmium or mercury poisoning, because the chelators can be selected to selectively bind to the metals and facilitate excretion.

Conventional chelators include, for example, aminopolyphosphonates, polycarboxylates, ethylenediaminetetraacetic acid (EDTA), diethylenetriaminepentaacetic acid (DTPA), and nitrilotriacetic acid (NTA). These and other conventional chelators, however, exhibit a number of undesirable properties. Some conventional chelators do not demonstrate adequate activity or stability across a wide pH and/or temperature range. Some conventional chelators exhibit an unacceptably high toxicity. Some conventional chelators do not exhibit adequate solubility in aqueous and/or organic solvents. Some conventional chelators have low biodegradability and present high environmental risk. Thus, the need exists for chelators that exhibit desirable properties, such as activity and/or stability across a wide pH and/or temperature range, low toxicity, adequate solubility, and/or high biodegradability.

Glycine derivatives, such as alanine-N,N-diacetonitrile, are a class of chelators that may exhibit these desirable properties. These chelators, which may be structural derivatives of the amino acid glycine, exhibit adequate activity suitable activity, stability, and biodegradability. Unfortunately, conventional processes, such as Strecker amino acid synthesis, for preparing glycine-derivative chelators are typically inefficient.

Thus, the need exists for improved processes for producing nitrile chelators and intermediates used to produce the nitrile chelators that demonstrate both efficiency and cost-effectiveness improvements. In particular, the need exists for producing glycine-derivative nitrile chelators using synergistic combinations of process conditions and without the need for a separate crystallization step. The resultant nitrile chelators and intermediates should have suitable (or improved) stability and activity across a wide pH and/or temperature range, low toxicity, and suitable biodegradability.

### SUMMARY

In one aspect, the present disclosure describes a process for preparing a nitrile intermediate, the process comprising: a first reaction step of reacting a tetra-amino compound with a hydrogen cyanide to form a reaction intermediate; and a second reaction step of reacting the reaction intermediate with the hydrogen cyanide and an aldehyde of the formula R—CHO, where R is (C₁-C₁₀)alkyl, (C₁-C₁₀)haloalkyl, (C₁-C₁₀)alkenyl, or (C₁-C₁₀)alkyl carboxylate, in an aqueous solution to form the nitrile intermediate. In some cases, the nitrile intermediate is formed at a yield greater than 75%. In some cases, the tetra-amino compound has a formula:

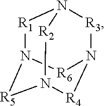

wherein R₁, R₂, R₃, R₄, R₅ and R₆ are independently (C₁-C₅)alkyl or (C₁-C₅)alkenyl, preferably (C₁-C₃)alkyl or (C₂-C₅)alkenyl. In some cases, the nitrile intermediate is alanine-N,N-dinitrile. In some cases, the reacting of the first reaction step comprises: providing a tetra-amino compound solution comprising the tetra-amino compound adjusting the pH of the tetra-amino compound solution to a pH ranging from 3.0 to 7.0; adding the hydrogen cyanide to the tetra-amino compound solution to form a first intermediate solution; heating and/or chilling the first intermediate solution to the first temperature; maintaining the first intermediate solution at the first temperature for up to 60 minutes; heating and/or chilling the heated first intermediate solution to the second temperature; and maintaining the first intermediate solution at the second temperature for up to 60 minutes. In some cases, the reacting of the second reaction step comprises: heating and/or chilling the first intermediate solution to the third temperature; adjusting the pH of the first intermediate solution to a pH ranging from 1.5 to 7.0; adding the hydrogen cyanide and the aldehyde to the first intermediate solution at the second temperature to form a second intermediate solution; and maintaining the second intermediate solution at the third temperature for from 15 to 250 minutes to form the nitrile intermediate. In some cases, the first reaction step and the second reaction step are carried out in the same vessel, i.e. one vessel. In some cases, the second reaction step comprises adding a nitrile intermediate seed to the reaction mixture. In some cases, the amount of nitrile intermediate seed added is less than 1% the theoretical yield of the nitrile intermediate. In some cases, the pH of the reaction mixture is reduced by at least 2.0, optionally be adding sulfuric acid. In some cases, the first reaction step is carried out at a pH from 3.0 to 7.0. In some cases, the second reaction step is carried out at a pH less than 5.0. In some cases, the first temperature is from 35° C. to 75° C.; and/or wherein the second temperature is from 50° C. to 100° C. In some cases, the second temperature is greater than the first temperature. In some cases, the third temperature is from 35° C. to 75° C. In some cases, the tetra-amino compound is 1,3,5,7-tetraazaadamantane. In some cases, R is (C₁-C₅)alkyl, and wherein R₁, R₂, R₃, R₄, R₅, and R₆ are independently (C₁-C₃)alkyl.

In some aspects, the processes described herein also comprise forming a glycine-N,N-diacetic acid derivative. In some cases, the glycine-N,N-diacetic acid derivative has a formula

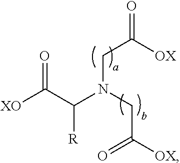

wherein: R is (C₁-C₁₀)alkyl, (C₁-C₁₀)haloalkyl, (C₁-C₁₀)alkenyl, or (C₁-C₁₀)alkyl carboxylate X is hydrogen, an alkali metal, an alkaline earth metal, or ammonium, a is from 0 to 5, and b is from 0 to 5; from the nitrile intermediate. In some cases, the forming the glycine-N,N-diacetic acid derivative comprises hydrolyzing the nitrile intermediate. In some cases, the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof. In some cases, the glycine-N,N-diacetic acid derivative is alanine-N,N-diacetic acid derivative. In some cases, the glycine-N,N-diacetic acid derivative is formed at a yield of at least 60%.

### DETAILED DESCRIPTION

### Introduction

As noted, the present disclosure describes specific reaction pathways and conditions for the production of nitrogen-containing chelator intermediates and the chelators, e.g., glycine derivatives, produced therefrom. In particular, the present disclosure describes a novel reaction scheme and synergistic combinations of operating parameters for the efficient production of a nitrile intermediate at a high yield and/or purity. The present inventors have developed the reaction scheme, including synergistic combinations of operating parameters, to provide a synthetic route for the production of nitrile intermediates at high yield and/or purity. The nitrile intermediate may then be further processed to produce the chelators at a high yield and/or a high purity.

The present disclosure describes a novel process for preparing a nitrile intermediate from a tetra-amino compound. In particular, in the processes described herein, the nitrile intermediate is formed by a two-step reaction of the tetra-amino compound with a hydrogen cyanide and an aldehyde (in an aqueous solution). The tetra-amino may have the structure:

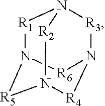

wherein R₁, R₂, R₃, R₄, R₅, and R₆ are independently (C₁-C₅) alkyl or (C₁-C₅) alkenyl. The aldehyde may have the formula R—CHO, where R is (C₁-C₁₀) alkyl, (C₁-C₁₀) haloalkyl, (C₁-C₁₀) alkenyl, or (C₁-C₁₀) alkyl carboxylate.

As discussed herein and as is demonstrated by the examples, the two-step reaction (optionally conducted as described herein) has been found to provide for unexpected improvements in overall yield and/or conversion.

In some cases, the reacting comprises controlling the addition of the reactants as well as the reaction conditions. For example, in some embodiments, the various reactants may be added and/or combined in a specific order, and the nitrile intermediate seed may be added at specific points in the overall reaction scheme. Controlling the reaction according to the present disclosure may provide for increased yield and/or purity of the nitrile intermediate.

In addition to the improvements in conversion and/or yield, the reaction pathways and conditions described herein may advantageously produce the nitrile intermediate in crystalline form, e.g., without the need for a separate crystallization step. Conventional processes such as Strecker amino acid synthesis, in contrast, are inefficient and produce a nitrile intermediate in non-crystalline form (e.g., as an emulsion), which then requires an inefficient crystallization step. This is typically accomplished by complicated mechanical means, such as complex agitation procedures. The crystallization step reduces the efficiency of the overall reaction and provides further opportunity for the loss of product and/or the formation of impurities. The elimination of the need for crystallizing beneficially increases the efficiency of the reaction. For example, without the need for a separate crystallization step, the nitrile intermediate can be produced and collected more quickly. The crystalline nitrile intermediate also better facilitates conversion to the nitrogen-containing chelator. In addition, removing the crystallization step reduces the costs associated with the production of the nitrogen-containing chelators.

In some cases, a nitrile intermediate seed may be employed during the reaction, (e.g., the seed is added to one or more of the (intermediate) reaction mixtures of the reacting step). The nitrile intermediate seed has been found to beneficially promote the formation of the nitrile intermediate in crystalline form. As a result, the (crystalline) nitrile intermediate is surprisingly produced with high purity and/or high yield. Conventional processes do not employ nitrile intermediate seeds, and, as such, require significant additional processing to achieve crystallization (e.g., controlled agitation to produce crystals).

As discussed in detail below, the reacting the tetra-amino compound, hydrogen cyanide, and aldehyde to form the nitrile intermediate may take many forms. The reacting may comprise combining the reactants in an aqueous solution and allowing the reaction to proceed. In some cases, the reactants are combined substantially simultaneously. In some cases, the reactants are combined in particular order.

### Reactants

### Tetra-Amino Compound

According to the present disclosure, a nitrile intermediate is produced from a tetra-amino compound (as a reactant). The structure of the tetra-amino compound is not particularly limited, and any organic compound having at least four amino functional groups may be used. For example, the tetra-amino compound may comprise a saturated or unsaturated carbon chain having four or more amino functional groups. In some embodiments, the amino functional groups may be moieties of a carbon chain that comprises one or more heteroatoms, such as oxygen, sulfur, or phosphorus. The tetra-amino compound may be aliphatic or aromatic and may be open-chain (e.g., branched-chain, straight-chain) or cyclic (e.g., polycyclic).

In some embodiments, the tetra-amino compound is an aliphatic polycycle having four amino functional groups. For example, the tetra-amino compound may have a chemical structure:

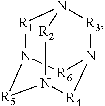

wherein R₁, R₂, R₃, R₄, R₅, and R₆ are independently (C₁-C₅)alkyl or (C₁-C₅)alkenyl, preferably (C₁-C₃)alkyl or (C₂-C₅)alkenyl. In some embodiments, the tetra-amino compound may have the above chemical structure, R₁, R₂, R₃, R₄, R₅, and R₆ are independently (C₁-C₃)alkyl. For example, R₁, R₂, R₃, R₄, R₅, and R₆ may be independently selected from a methylene group, an ethylene group, an n-propylene group, or an isopropylene group. In some embodiments, the tetra-amino compound may have the above chemical structure, wherein at least one of R₁, R₂, R₃, R₄, R₅, and R₆ is a methylene group, e.g., at least two, at least three, or at least four of R₁, R₂, R₃, R₄, R₅, and R₆ are methylene groups. Exemplary tetra-amino compounds according to the above chemical structure include tetraazaadamantane (e.g., 1,3,5,7-tetraazaadamantane), methyl-tetraazaadamantane, dimethyl-tetraazaadamantane, trimethyl-tetraazaadamantane, tetramethyl-tetraazaadamantane, ethyl-tetraazaadamantane, diethyl-tetraazaadamantane, triethyl-tetraazaadamantane, tetraethyl-tetraazaadamantane, ethyl-methyl-tetraazaadamantane, propyl-tetraazaadamantane, dipropyl-tetraazaadamantane, tripropyl-tetraazaadamantane, tetrapropyl-tetraazaadamantane, and methyl-propyl-tetraazaadamantane.

In some embodiments, the tetra-amino compound may be dissolved in a solution, e.g., the tetra-amino compound may be a component of a tetra-amino compound solution (discussed in detail below). For example, the tetra-amino compound may be mixed with and/or dissolved in a solvent. The composition of the tetra-amino compound solution is not particularly limited and may be any solution of the tetra-amino compound. In some embodiments, for example, the tetra-amino compound solution may comprise the tetra-amino compound dissolved in an aqueous solvent, e.g., water, an organic solvent, or a solvent system of both aqueous and organic solvents.

### Aldehyde

The aldehyde may vary widely and many suitable aldehydes are known. In particular, the aldehyde may have a chemical formula R—CHO, where R is (C₁-C₁₀)alkyl, (C₁-C₁₀)haloalkyl, (C₁-C₁₀)alkenyl, or (C₁-C₁₀)alkyl carboxylate. In some embodiments, R of the aldehyde is (C₁-C₁₀)alkyl, e.g., (C₁-C₉)alkyl, (C₁-C₅)alkyl, (C₁-C₇)alkyl, (C₁-C₆)alkyl, or (C₁-C₅)alkyl. In some embodiments, R of the aldehyde is (C₁-C₁₀)haloalkyl, e.g., (C₁-C₉)haloalkyl, (C₁-C₅)haloalkyl, (C₁-C₇)haloalkyl, (C₁-C₆)haloalkyl, or (C₁-C₅)haloalkyl. In some embodiments, R of the aldehyde is (C₁-C₁₀)alkenyl, e.g., (C₂-C₁₀)alkenyl, (C₁-C₉)alkenyl, (C₂-C₉)alkenyl, (C₁-C₅)alkenyl, (C₂-C₅)alkenyl, (C₁-C₇)alkenyl, (C₂-C₇)alkenyl, (C₁-C₆)alkenyl, (C₂-C₆)alkenyl, (C₁-C₅)alkenyl or (C₂-C₅)alkenyl. In some embodiments, R of the aldehyde is (C₁-C₁₀)alkyl carboxylate, e.g., (C₁-C₉)alkyl carboxylate, (C₁-C₅)alkyl carboxylate, (C₁-C₇)alkyl carboxylate, (C₁-C₆)alkyl carboxylate, or (C₁-C₅)alkyl carboxylate. For example, the aldehyde may comprise a saturated or unsaturated, straight or branched carbon chain, e.g., a terminal carbonyl functional group. Exemplary aldehydes include acetaldehyde, propionaldehyde, butyraldehyde, pentanal, propenal, butenal, formyl ethanoic acid, formyl propionic acid, and formyl butanoic acid.

As noted above, the order of the addition of the aldehyde to the other reactants may vary widely. In some cases, an aldehyde is added to and/or reacted with the tetra-amino compound (optionally in a heated tetra-amino compound solution) to form a first intermediate solution. For example, the aldehyde may be added to the tetra-amino compound solution, and/or the tetra-amino compound may be added to aldehyde. In some cases, the aldehyde may be added to a solution comprising the tetra-amino compound and the hydrogen cyanide.

In some embodiments, the nitrile intermediate seed is added to and/or reacted with the first intermediate solution to form a second intermediate solution. As noted, the nitrile intermediate seed may be added at other points in the reaction scheme, examples of which are discussed in more detail herein.

The amount of aldehyde used in the reaction, e.g., the amount aldehyde present in the first intermediate solution or the second intermediate solution, is not particularly limited. The amount of aldehyde used may be based on the amount of tetra-amino compound. In some embodiments, for example, an amount of aldehyde is added such that the molar ratio of the aldehyde to the tetra-amino compound is from 0.1:1 to 10:1, e.g., from 0.1:1 to 8:1, from 0.1:1 to 6:1, from 0.1:1 to 4:1, from 0.1:1 to 3:1, from 0.2:1 to 10:1, from 0.2:1 to 8:1, from 0.2:1 to 6:1, from 0.2:1 to 4:1, from 0.2:1 to 3:1, from 0.4:1 to 10:1, from 0.4:1 to 8:1, from 0.4:1 to 6:1, from 0.4:1 to 4:1, from 0.4:1 to 3:1, from 0.5:1 to 10:1, from 0.5:1 to 8:1, from 0.5:1 to 6:1, from 0.5:1 to 4:1, from 0.5:1 to 3:1, from 0.8:1 to 10:1, from 0.8:1 to 8:1, from 0.8:1 to 6:1, from 0.8:1 to 4:1, or from 0.8:1 to 3:1. In terms of lower limits, the molar ratio of the aldehyde to the tetra-amino compound may be greater than 0.1:1, e.g., greater than 0.2:1, greater than 0.4:1, greater than 0.5:1, or greater than 0.8:1. In terms of upper limits, the molar ratio of the aldehyde to the tetra-amino compound may be less than 10:1, e.g., less than 8:1, less than 6:1, less than 4:1, or less than 3:1.

### Hydrogen Cyanide

The hydrogen cyanide (HCN) is added to and/or reacted with one or more of the other reactants. The hydrogen cyanide may be combined with the tetra-amino compound before or after the aldehyde is introduced. In some embodiments, the aldehyde and the hydrogen cyanide are combined with the tetra-amino compound at substantially the same time, e.g., simultaneously or within several minutes of each other. In some embodiments, the hydrogen cyanide is added to and/or reacted with the tetra-amino compound solution.

The HCN, in some instances, may be employed in the form of a solution comprising HCN and the solution may be reacted with the tetra-amino compound (and the aldehyde) as described herein.

The amount of hydrogen cyanide used in the reaction, e.g., the amount hydrogen cyanide added to the tetra-amino compound solution, is not particularly limited. The amount of hydrogen cyanide used may be based on the amount of tetra-amino compound. In some embodiments, for example, an amount of hydrogen cyanide is added such that the molar ratio of the hydrogen cyanide to the tetra-amino compound is from 0.1:1 to 10:1, e.g., from 0.1:1 to 8:1, from 0.1:1 to 6:1, from 0.1:1 to 4:1, from 0.1:1 to 3:1, from 0.2:1 to 10:1, from 0.2:1 to 8:1, from 0.2:1 to 6:1, from 0.2:1 to 4:1, from 0.2:1 to 3:1, from 0.4:1 to 10:1, from 0.4:1 to 8:1, from 0.4:1 to 6:1, from 0.4:1 to 4:1, from 0.4:1 to 3:1, from 0.5:1 to 10:1, from 0.5:1 to 8:1, from 0.5:1 to 6:1, from 0.5:1 to 4:1, from 0.5:1 to 3:1, from 0.8:1 to 10:1, from 0.8:1 to 8:1, from 0.8:1 to 6:1, from 0.8:1 to 4:1, or from 0.8:1 to 3:1. In terms of lower limits, the molar ratio of the hydrogen cyanide to the tetra-amino compound may be greater than 0.1:1, e.g., greater than 0.2:1, greater than 0.4:1, greater than 0.5:1, or greater than 0.8:1. In terms of upper limits, the molar ratio of the hydrogen cyanide to the tetra-amino compound may be less than 10:1, e.g., less than 8:1, less than 6:1, less than 4:1, or less than 3:1.

### Nitrile Intermediate Seed

In some embodiments, the disclosed processes may employ a nitrile intermediate seed during the reaction. The timing of the addition of the nitrile intermediate seed to one or more of the reaction mixtures may vary. The addition of the nitrile intermediate seed has surprisingly been found to greatly improve the preparation of the nitrile intermediate, and subsequently the glycine derivative. In particular, the addition of the nitrile intermediate seed supports the formation of the nitrile intermediate (e.g., by the reaction of the dinitrile compound, the aldehyde, and the hydrogen cyanide) in crystalline form. Said another way, in some cases, the processes described herein produce crystalline nitrile intermediate due to the addition of the nitrile intermediate seed during the reaction. Furthermore, the formation of crystals in situ during the reaction contributes to improved yield and purity of the nitrile intermediate produced by the reaction.

Generally, the nitrile intermediate seed is an organic compound having at least two nitrile, or cyano, functional groups and at least one carboxyl functional group. Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile

In some embodiments, the chemical composition of the nitrile intermediate seed may be defined in relation to the nitrile intermediate to be formed by the reaction. For example, the nitrile intermediate seed may comprise substantially the same chemical structure (e.g., the same or slightly modified chemical structure) as the nitrile intermediate. Thus, any composition of the nitrile intermediate (discussed in detail below) may be used as the nitrile intermediate seed. The nitrile intermediate seed may be solid of the nitrile intermediate or may be a liquid solution comprising the nitrile intermediate. In some embodiments, for example, the nitrile intermediate seed is a solid crystal of the nitrile intermediate.

In the processes described herein, the nitrile intermediate seed is added to a reaction mixture before and/or during the first reaction step or second reaction step. In some embodiments, the nitrile intermediate seed may combined with the dinitrile compound (e.g., the nitrile intermediate seed may be added to the dinitrile compound solution before the addition of both the aldehyde and the hydrogen cyanide). In some embodiments, the nitrile intermediate seed is added to the reaction mixture after the addition of the aldehyde (and before addition of the hydrogen cyanide). For example, the nitrile intermediate seed may be combined with the first intermediate solution (e.g., comprising the dinitrile compound and the aldehyde) to produce a second intermediate solution. In some embodiments, the nitrile intermediate seed is added to the reaction mixture after the addition of the hydrogen cyanide. In some embodiments, the nitrile intermediate seed is added to the reaction mixture at substantially the same time as the aldehyde and/or the hydrogen cyanide.

In some cases, only a small amount of the nitrile intermediate seed is required to produce the effects described herein, but larger amounts are contemplated. The amount of nitrile intermediate seed added to the reaction mixture may be described by reference to the theoretical yield of the nitrile intermediate by the reaction. In some embodiments, the amount of nitrile intermediate seed added to the reaction mixture is less than 1% of the theoretical yield of the nitrile intermediate by the reaction, e.g., less than 0.8%, less than 0.5%, less than 0.2%, less than 0.1%, or less than 0.08%. In terms of lower limits, the amount of nitrile intermediate added to the reaction mixture is greater than 0.0001% the theoretical yield of the nitrile intermediate by the reaction, e.g., greater than 0.0005%, greater than 0.001%, greater than 0.005%, or greater than 0.008%.

The amount of nitrile intermediate seed added to the reaction mixture may also be described by reference to the weight percentage of the nitrile intermediate seed in the reaction mixture (e.g., the weight percentage of the nitrile intermediate seed in the second intermediate solution). In some embodiments, the second intermediate solution comprises from 0.001 wt. % to 1 wt. % of the nitrile intermediate seed, e.g., from 0.001 wt. % to 0.5 wt. %, from 0.001 wt. % to 0.1 wt. %, from 0.001 wt. % to 0.1 wt. %, from 0.001 wt. % to 0.08 wt. %, from 0.005 wt. % to 1 wt. %, from 0.005 wt. % to 0.5 wt. %, from 0.005 wt. % to 0.1 wt. %, from 0.005 wt. % to 0.1 wt. %, from 0.005 wt. % to 0.08 wt. %, from 0.008 wt. % to 1 wt. %, from 0.008 wt. % to 0.5 wt. %, from 0.008 wt. % to 0.1 wt. %, from 0.008 wt. % to 0.1 wt. %, from 0.008 wt. % to 0.08 wt. %, from 0.01 wt. % to 1 wt. %, from 0.01 wt. % to 0.5 wt. %, from 0.01 wt. % to 0.1 wt. %, from 0.01 wt. % to 0.1 wt. %, or from 0.01 wt. % to 0.08 wt. %. In terms of upper limits, the second intermediate solution may comprise less than 1 wt. % nitrile intermediate seed, e.g., less than 0.5 wt. %, less than 0.1 wt. %, or less than 0.08 wt. %.

### Two-Step Reaction

As noted above, the reaction of the processes described herein is carried out in two steps. The utilization of the two-step mechanism provides for the unexpected benefits mentioned herein. In particular, the two-step reaction scheme, including the operating parameters described herein, produces nitrile intermediates at high yield and/or purity. In a first reaction step, the tetra-amino compound is allowed to react with the hydrogen cyanide. This first reaction step produced a reaction intermediate, which may or may not be separated or purified. In a second reaction step, the reaction intermediate is allowed to react with the aldehyde and hydrogen cyanide to produce the nitrile intermediate. Carrying out the reaction in two steps, as described herein, efficiently produces the reaction intermediate (at least in part) before addition of the aldehyde. This improves the yield of the ultimate nitrile intermediate.

Each reaction step may comprise controlling the addition of the reactants as well as the reaction conditions. For example, in some embodiments, the various reactants may be added and/or combined in a specific order, and the nitrile intermediate seed may be added at specific points in the overall reaction scheme. The reaction conditions described herein may further improve the production of the nitrile intermediate, e.g., the purity and/or yield of the nitrile intermediate. In particular, the present disclosure provides temperature and pH conditions, which the present inventors have surprisingly found produce the purity and/or the yield of the nitrile intermediate by the reaction described herein.

Controlling the reaction according to the present disclosure may provide for increased yield and/or purity of the nitrile intermediate.

### First Reaction Step

The process of the present disclosure includes a first reaction step of reacting the tetra-amino compound with hydrogen cyanide to form a reaction intermediate. In one embodiment, the hydrogen cyanide is reacted a first temperature and heated to a second temperature that is higher than the first temperature.

In some embodiments, the first reaction step comprises providing a tetra-amino compound solution comprising the tetra-amino compound. For example, the process may include dissolving the tetra-amino compound in a solvent to prepare the tetra-amino compound solution. The composition of the tetra-amino compound solution is not particularly limited and may be any solution of the tetra-amino compound. In some embodiments, for example, the tetra-amino compound solution may comprise the tetra-amino compound dissolved in an aqueous solvent, e.g., water. In some embodiments, the tetra-amino compound solution may comprise the tetra-amino compound dissolved in an organic solvent. In some embodiments, the tetra-amino compound solution is a solution of the tetra-amino dissolved in a solvent system of both aqueous and organic solvents.

The concentration of the tetra-amino compound solution is not particularly limited. In some embodiments, the tetra-amino compound solution comprises from 1 wt. % to 50 wt. % of the tetra-amino compound, e.g., from 1 wt. % to 45 wt. %, from 1 wt. % to 40 wt. %, from 1 wt. % to 35 wt. %, from 1 wt. % to 30 wt. %, from 4 wt. % to 50 wt. %, from 4 wt. % to 45 wt. %, from 4 wt. % to 40 wt. %, from 4 wt. % to 35 wt. %, from 4 wt. % to 30 wt. %, from 8 wt. % to 50 wt. %, from 8 wt. % to 45 wt. %, from 8 wt. % to 40 wt. %, from 8 wt. % to 35 wt. %, from 8 wt. % to 30 wt. %, from 10 wt. % to 50 wt. %, from 10 wt. % to 45 wt. %, from 10 wt. % to 40 wt. %, from 10 wt. % to 35 wt. %, or from 10 wt. % to 30 wt. %. In terms of lower limits, the tetra-amino compound solution may comprise greater than 1 wt. % of the tetra-amino compound, e.g., greater than 4 wt. %, greater than 8 wt. %, or greater than 10 wt. %. In terms of upper limits, the tetra-amino compound solution may comprise less than 40 wt. % of the tetra-amino compound, e.g., less than 45 wt. %, less than 40 wt. %, less than 35 wt. %, or less than 30 wt. %.

Without being limited by theory, the tetra-amino compound solution may be provided for the reaction at any temperature or may be heated to a target temperature. In some embodiments, the tetra-amino compound solution is provided at room temperature. In some embodiments, the tetra-amino compound is from about 10° C. to about 30° C., e.g., from about 10° C. to about 29° C., from about 10° C. to about 28° C., from about 10° C. to about 27° C., from about 10° C. to about 26° C., from about 10° C. to about 25° C., from about 12° C. to about 30° C., from about 12° C. to about 29° C., from about 12° C. to about 28° C., from about 12° C. to about 27° C., from about 12° C. to about 26° C., from about 12° C. to about 25° C., from about 14° C. to about 30° C., from about 14° C. to about 29° C., from about 14° C. to about 28° C., from about 14° C. to about 27° C., from about 14° C. to about 26° C., from about 14° C. to about 25° C., from about 18° C. to about 30° C., from about 18° C. to about 29° C., from about 18° C. to about 28° C., from about 18° C. to about 27° C., from about 18° C. to about 26° C., from about 18° C. to about 25° C., from about 20° C. to about 30° C., from about 20° C. to about 29° C., from about 20° C. to about 28° C., from about 20° C. to about 27° C., from about 20° C. to about 26° C., from about 20° C. to about 25° C., from about 22° C. to about 30° C., from about 22° C. to about 29° C., from about 22° C. to about 28° C., from about 22° C. to about 27° C., from about 22° C. to about 26° C., or from about 22° C. to about 25° C.

In some embodiments, the first reaction step comprises adjusting the pH of the tetra-amino compound solution. The acidity and/or alkalinity of the reactants (and/or the reaction mixture and/or the various intermediate mixtures) can greatly affect the progress of the reaction described herein. In particular, the reaction of the present disclosure may require an acidic environment (e.g., pH less than 7), and so it may be preferable to adjust the pH of the tetra-amino compound solution prior to the addition of and/or mixture with other reactants. In some embodiments, the tetra-amino compound solution is provided at an approximately neutral pH, e.g., a pH ranging from 3.0 to 9, e.g., from 6 to 8, from 6.5 to 7.5, or from 6.75 to 7.25. Thus, in some cases, the reacting comprises adjusting the pH of the dinitrile compound solution. In some embodiments, the pH can be modified by the addition of a mineral acid, e.g., hydrochloric acid, nitric acid, phosphoric acid, sulfuric acid, boric acid, hydrofluoric acid, hydro bromic acid, per chloric acid, or hydroiodic acid.

In some embodiments, the tetra-amino compound solution is adjusted to a pH ranging from 3.0 to 7.0, e.g., from 3.0 to 6.8, from 3.0 to 6.6, from 3.0 to 6.4, from 3.0 to 6.2, from 3.0 to 6.0, from 3.8 to 7.0, from 3.8 to 6.8, from 3.8 to 6.6, from 3.8 to 6.4, from 3.8 to 6.2, from 3.8 to 6.0, from 4.0 to 7.0, from 4.0 to 6.8, from 4.0 to 6.6, from 4.0 to 6.4, from 4.0 to 6.2, from 4.0 to 6.0, from 4.2 to 7.0, from 4.2 to 6.8, from 4.2 to 6.6, from 4.2 to 6.4, from 4.2 to 6.2, from 4.2 to 6.0, from 4.5 to 7.0, from 4.5 to 6.8, from 4.5 to 6.6, from 4.5 to 6.4, from 4.5 to 6.2, or from 4.5 to 6.3.

In some embodiments, the first reaction step comprises adding the hydrogen cyanide to the tetra-amino compound solution to form a first intermediate solution. The method of adding the hydrogen cyanide is not particularly limited. In some cases, for example, the hydrogen cyanide may be added to the tetra-amino compound solution by a syringe, e.g., a sub-surface syringe. In one embodiment, the hydrogen cyanide is added at a rate from 0.01 g/min to 1 g/min, e.g., from 0.02 g/min to 0.8 g/min, from 0.05 g/min to 0.6 g/min, or from 0.08 g/min to 0.4 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.8 g/min, less than 0.6 g/min, or less than 0.4 g/min.

The temperature of the hydrogen cyanide added to the tetra-amino compound solution is not particularly limited. In some cases, the first reaction step comprises adjusting the temperature of the hydrogen cyanide (or the solution containing the hydrogen cyanide). In some embodiments, the hydrogen cyanide is heated or chilled (e.g., before addition to the tetra-amino compound solution) to a temperature from 0° C. to 40° C., e.g., from 1° C. to 35° C., from 2° C. to 30° C., or from 3° C. to 25° C. Similarly, the pH of the hydrogen cyanide is not particularly limited. In some cases, the first reaction step comprises modifying (e.g., controlling and/or adjusting) the pH of the hydrogen cyanide (e.g., before addition to the tetra-amino compound solution) to a pH from 0.5 to 9.

In some embodiments, the first reaction step comprises heating and/or chilling the first intermediate solution to a first temperature. For example, the first intermediate solution may be heated during and/or after the addition of the hydrogen cyanide. In some embodiments, the first temperature is from 35° C. to 75° C., e.g. from 35° C. to 72° C., from 35° C. to 70° C., from 35° C. to 68° C., from 35° C. to 65° C., from 38° C. to 75° C., from 38° C. to 72° C., from 38° C. to 70° C., from 38° C. to 68° C., from 38° C. to 65° C., from 40° C. to 75° C., from 40° C. to 72° C., from 40° C. to 70° C., from 40° C. to 68° C., from 40° C. to 65° C., from 42° C. to 75° C., from 42° C. to 72° C., from 42° C. to 70° C., from 42° C. to 68° C., or from 42° C. to 65° C. In terms of upper limits, the first temperature may be less than 75° C., e.g., less than 72° C., less than 70° C., less than 68° C., or less than 65° C. In terms of lower limits, the first temperature may be greater than 35° C., e.g., greater than 38° C., greater than 40° C., or greater than 42° C. In some embodiments, the first reaction step comprises maintaining the first intermediate solution at the first temperature for up to 60 minutes, e.g., up to 50 minutes, up to 40 minutes, or up to 30 minutes.

In some embodiments, the first reaction step comprises heating and/or chilling the first intermediate solution to a second temperature. For example, the first intermediate solution may be heated during and/or after the addition of the hydrogen cyanide. In some embodiments, the second temperature is from 50° C. to 100° C., e.g. from 50° C. to 95° C., from 50° C. to 90° C., from 50° C. to 85° C., from 50° C. to 80° C., from 55° C. to 100° C., from 55° C. to 95° C., from 55° C. to 90° C., from 55° C. to 85° C., from 55° C. to 80° C., from 60° C. to 100° C., from 60° C. to 95° C., from 60° C. to 90° C., from 60° C. to 85° C., from 60° C. to 80° C., from 65° C. to 100° C., from 65° C. to 95° C., from 65° C. to 90° C., from 65° C. to 85° C., or from 65° C. to 80° C. In terms of upper limits, the second temperature may be less than 100° C., e.g., less than 95° C., less than 90° C., less than 85° C., or less than 80° C. In terms of lower limits, the second temperature may be greater than 50° C., e.g., greater than 55° C., greater than 60° C., or greater than 65° C. In some embodiments, the first reaction step comprises maintaining the first intermediate solution at the second temperature for up to 60 minutes, e.g., up to 50 minutes, up to 40 minutes, or up to 30 minutes.

In some embodiments, the first reaction step comprises some combination of the above-described conditions and parameters. Said another way, the first reaction step may comprise any combination of the above described temperature, pH, and mixing parameters. In some embodiments, for example, the first reaction step may include providing a tetra-amino compound solution comprising the tetra-amino compound, adjusting the pH of the tetra-amino compound solution to a pH ranging from 3.0 to 7.0, adding the hydrogen cyanide to the tetra-amino compound solution to form a first intermediate solution, heating the first intermediate solution a first temperature, maintaining the first intermediate solution at the first temperature for up to 15 minutes, maintaining the first intermediate solution at the first temperature for up to 15 minutes, heating the first intermediate solution to the second temperature, and/or maintaining the first intermediate solution at the second temperature for up to 60 minutes.

### Reaction Intermediate

The first reaction step may produce a reaction intermediate. The reaction intermediate is not particularly limited and will vary with the reactants (e.g., the tetra-amino compound). Generally, the reaction intermediate is a dinitrile compound, e.g., an organic compound having at least two nitrile, or cyano (—C≡N), functional groups. For example, the dinitrile compound may comprise a saturated or unsaturated carbon chain having two or more nitrile functional groups. In some embodiments, the nitrile functional groups may be moieties of a carbon chain that comprises one or more heteroatoms, such as oxygen, nitrogen, sulfur, or phosphorus. In some embodiments, the reaction intermediate is a compound having the chemical structure:

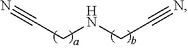

wherein a is from 0 to 5 and b is from 0 to 5. In some embodiments, the reaction intermediate is a dinitrile compound having the above chemical structure, wherein a is 1, and b is 0, 1, 2, 3, 4, or 5. In some embodiments, the dinitrile compound may have the above chemical structure, wherein a is 1 or 2, and b is 0, 1, 2, 3, or 4. In some embodiments, the dinitrile compound may have the above chemical structure, wherein a is 1, 2, or 3, and b is 1, 2, or 3. Exemplary dinitrile compounds according to the above chemical structure include ((cyanomethyl)amino)acetonitrile, ((cyanomethyl)amino)propanenitrile, ((cyanomethyl)amino)butanenitrile, ((cyanomethyl)amino)pentanenitrile, ((cyanoethyl)amino)acetonitrile, ((cyanoethyl)amino)propanenitrile, ((cyanoethyl)amino)butanenitrile, ((cyanoethyl)amino)pentanenitrile, ((cyanopropyl)amino)acetonitrile, ((cyanopropyl)amino)propanenitrile, ((cyanopropyl)amino)butanenitrile, ((cyanopropyl)amino)pentanenitrile, ((cyanobutyl)amino)acetonitrile, ((cyanobutyl)amino)propanenitrile, ((cyanobutyl)amino)butanenitrile, ((cyanobutyl)amino)pentanenitrile, ((cyanopropyl)amino)acetonitrile, ((cyanopropyl)amino)propanenitrile, ((cyanopropyl)amino)butanenitrile, and ((cyanopropyl)amino)pentanenitrile.

### Second Reaction Step

The process of the present disclosure includes a second reaction step of reacting the reaction intermediate with hydrogen cyanide and the aldehyde at a first temperature followed by a second temperature to form the nitrile intermediate. In some cases, the reaction intermediate reacts with aldehyde and/or hydrogen cyanide by Strecker synthesis to produce the nitrile intermediate. Because the components of the first intermediate solution (e.g., the tetra-amino compound, the hydrogen cyanide, the reaction intermediate) may be reactants in the second step, the second reaction step may be carried out in the same vessel as the first reaction step.

In some embodiments, the second reaction step comprises heating and/or chilling the first intermediate solution to a third temperature. For example, the first intermediate solution may be heated during and/or after the completion of the first reaction step. In some embodiments, the third temperature is from 35° C. to 75° C., e.g. from 35° C. to 72° C., from 35° C. to 70° C., from 35° C. to 68° C., from 35° C. to 65° C., from 38° C. to 75° C., from 38° C. to 72° C., from 38° C. to 70° C., from 38° C. to 68° C., from 38° C. to 65° C., from 40° C. to 75° C., from 40° C. to 72° C., from 40° C. to 70° C., from 40° C. to 68° C., from 40° C. to 65° C., from 42° C. to 75° C., from 42° C. to 72° C., from 42° C. to 70° C., from 42° C. to 68° C., or from 42° C. to 65° C. In terms of upper limits, the third temperature may be less than 75° C., e.g., less than 72° C., less than 70° C., less than 68° C., or less than 65° C. In terms of lower limits, the third temperature may be greater than 35° C., e.g., greater than 38° C., greater than 40° C., or greater than 42° C.

In some embodiments, the second reaction step comprises adjusting the pH of the first intermediate solution (e.g., produced in the first reaction step). As noted above, pH can be modified by the addition of a mineral acid, e.g., hydrochloric acid, nitric acid, phosphoric acid, sulfuric acid, boric acid, hydrofluoric acid, hydrobromic acid, perchloric acid, or hydroiodic acid. In some embodiments, the pH of the first intermediate solution is adjusted to a pH ranging from 1.5 to 7.0, e.g., from 1.5 to 6.5, from 1.5 to 6.0, from 1.5 to 5.5, from 2.0 to 7.0, from 2.0 to 6.5, from 2.0 to 6.0, from 2.0 to 5.5, from 2.5 to 7.0, from 2.5 to 6.5, from 2.5 to 6.0, from 2.5 to 5.5, from 3.0 to 7.0, from 3.0 to 6.5, from 3.0 to 6.0, from 3.0 to 5.5.

In some embodiments, the second reaction step comprises adding the aldehyde (or a solution containing the aldehyde) and additional hydrogen cyanide to the first intermediate solution, e.g., to form a second intermediate solution.

The method of adding the aldehyde is not particularly limited. In some cases, for example, the aldehyde may be added to the tetra-amino compound solution by a syringe, e.g., a sub-surface syringe. In one embodiment, the aldehyde is added at a rate from 0.05 mL/min to 10 mL/min, e.g., from 0.1 mL/min to 8 mL/min, from 0.15 mL/min to 5 mL/min, or from 0.2 mL/min to 2 mL/min. In terms of lower limits, the addition rate may be greater than 0.05 mL/min, e.g., greater than 0.1 mL/min, greater than 0.15 mL/min, or greater than 0.2 mL/min. In terms of upper limits, the addition rate may be less than 10 mL/min, e.g., less than 8 mL/min, less than 5 mL/min, less than 2 mL/min, or less than 1 mL/min.

The temperature of the aldehyde added to the first intermediate solution is not particularly limited. In some cases, the second reaction step comprises adjusting the temperature of the aldehyde (or of the solution containing the aldehyde) before addition to the first intermediate solution. In some embodiments, the aldehyde is heated or chilled to a temperature from 1° C. to 40° C., e.g., from 2° C. to 35° C., from 3° C. to 30° C., or from 4° C. to 25° C. Similarly, the pH of the aldehyde is not particularly limited. In some cases, the second reaction comprises modifying (e.g., controlling and/or adjusting) the pH of the aldehyde (e.g., before combination with the tetra-amino compound solution) to a pH from 0.5 to 9.

Likewise, the method of adding the hydrogen cyanide is not particularly limited. In some cases, for example, the hydrogen cyanide may be added to the first intermediate solution by a syringe, e.g., a sub-surface syringe. In one embodiment, the hydrogen cyanide is added at a rate from 0.01 g/min to 1 g/min, e.g., from 0.02 g/min to 0.8 g/min, from 0.05 g/min to 0.6 g/min, or from 0.08 g/min to 0.4 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.8 g/min, less than 0.6 g/min, or less than 0.4 g/min.

The temperature of the hydrogen cyanide added to the first intermediate solution is not particularly limited. In some cases, the first reaction step comprises adjusting the temperature of the hydrogen cyanide (or the solution containing the hydrogen cyanide). In some embodiments, the hydrogen cyanide is heated or chilled to a temperature from 0° C. to 40° C., e.g., from 1° C. to 35° C., from 2° C. to 30° C., or from 3° C. to 25° C. Similarly, the pH of the hydrogen cyanide is not particularly limited. In some cases, the first reaction step comprises modifying (e.g., controlling and/or adjusting) the pH of the hydrogen cyanide (e.g., before addition to the first intermediate solution) to a pH from 0.5 to 9.

In some cases, the second reaction step may comprise adding the nitrile intermediate seed to the reaction mixture (e.g., the first intermediate solution and/or the second intermediate solution). As noted above, a relatively small amount of nitrile intermediate seed is added to the reaction mixture. In some embodiments, the amount of nitrile intermediate seed added to the reaction mixture is less than 1% of the theoretical yield of the nitrile intermediate by the reaction, e.g., less than 0.8%, less than 0.5%, less than 0.2%, less than 0.1%, or less than 0.08%. In terms of lower limits, the amount of nitrile intermediate added to the reaction mixture is greater than 0.0001% the theoretical yield of the nitrile intermediate by the reaction, e.g., greater than 0.0005%, greater than 0.001%, greater than 0.005%, or greater than 0.008%.

In some embodiments, the aldehyde and the hydrogen cyanide are added to the first intermediate solution at the first temperature, as described above. In some embodiments, the first intermediate solution may be heated to the first temperature before, during, and/or after the addition of the aldehyde and/or the hydrogen cyanide. In some embodiments, the second intermediate solution is maintained at the first and/or third temperature to allow the reaction to progress. For example, the second intermediate solution may be maintained at the first and/or third temperature for from 15 minutes to 250 minutes, e.g., from 15 minutes to 240 minutes, from 15 minutes to 240 minutes, from 15 minutes to 220 minutes, from 30 minutes to 250 minutes, from 30 minutes to 240 minutes, from 30 minutes to 240 minutes, from 30 minutes to 220 minutes, from 45 minutes to 250 minutes, from 45 minutes to 240 minutes, from 45 minutes to 240 minutes, from 45 minutes to 220 minutes, from 60 minutes to 250 minutes, from 60 minutes to 240 minutes, from 60 minutes to 240 minutes, from 60 minutes to 220 minutes, from 75 minutes to 250 minutes, from 75 minutes to 240 minutes, from 75 minutes to 240 minutes, or from 75 minutes to 220 minutes.

In some embodiments, the second reaction step comprises some combination of the above-described conditions and parameters. Said another way, the second reaction step may comprise any combination of the above described temperature, pH, and mixing parameters. In some embodiments, for example, the second reaction step may include adjusting the pH of the first intermediate solution to a pH ranging from 1.5 to 7.0, adding the hydrogen cyanide and the aldehyde to the first intermediate solution at the second temperature to form a second intermediate solution; and maintaining the second intermediate solution at the second temperature for from 30 to 250 minutes to form the nitrile intermediate.

In some cases, the second reaction step also includes cooling the second intermediate solution. This causes the nitrile intermediate produced by the process to form crystals, which may be harvested (e.g., filtered). In some embodiments, for example, the second intermediate solution is cooled to a temperature less than 25° C., e.g., less than 20° C., less than 15° C., or less than 10° C.

### Product, Nitrile Intermediate

As discussed above, the first reaction step and the second reaction step produce a nitrile intermediate. The nitrile intermediate is not particularly limited and will vary with the tetra-amino compound. Generally, the nitrile intermediate is an organic compound having at least two nitrile, or cyano, functional groups and at least one carboxyl functional group. In some embodiments, the nitrile intermediate is a compound having the chemical structure:

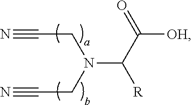

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In some embodiments, the nitrile intermediate may have the above chemical structure, wherein a is 1, and b is 0, 1, 2, 3, 4, or 5. In some embodiments, the nitrile intermediate may have the above chemical structure, wherein a is 1 or 2, and b is 0, 1, 2, 3, or 4. In some embodiments, the nitrile intermediate may have the above chemical structure, wherein a is 1, 2, or 3, and b is 1, 2, or 3. In some embodiments, R of the nitrile intermediate is (C₁-C₁₀)alkyl, e.g., (C₁-C₉)alkyl, (C₁-C₅)alkyl, (C₁-C₇)alkyl, (C₁-C₆)alkyl, or (C₁-C₅)alkyl. In some embodiments, R of the nitrile intermediate is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. In some embodiments, R of the nitrile intermediate is (C₁-C₁₀)alkenyl, e.g., (C₂-C₁₀)alkenyl, (C₁-C₉)alkenyl, (C₂-C₉)alkenyl, (C₁-C₈)alkenyl, (C₂-C₈)alkenyl, (C₁-C₇)alkenyl, (C₂-C₇)alkenyl, (C₁-C₆)alkenyl, (C₂-C₆)alkenyl, (C₁-C₅)alkenyl or (C₂-C₅)alkenyl. In some embodiments, R of the nitrile intermediate is (C₁-C₁₀)alkyl carboxylate, e.g., (C₁-C₉)alkyl carboxylate, (C₁-C₈)alkyl carboxylate, (C₁-C₇)alkyl carboxylate, (C₁-C₆)alkyl carboxylate, or (C₁-C₅)alkyl carboxylate. In particular, a and b may correspond to their respective values in the tetra-amino compound, and R may correspond to its respective value in the aldehyde.

Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile.

As has been discussed, the processes described herein produce the nitrile intermediate in crystalline form. Said another way, crystals of the nitrile intermediate are produced by the described processes, in particular without need for a separate crystallization step. Furthermore, the nitrile intermediate does not form an emulsion and therefore does not require additional mechanical processing (e.g., agitation) to separate. The formation of the nitrile intermediate in crystalline form increases the efficiency of the production process by removing the need for an additional step (and eliminating the time and cost associated therewith).

The two-step reaction scheme described herein favorably results in efficient nitrile intermediate production. Said another way, the process produces the nitrile intermediate at high yield. In some embodiments, the nitrile intermediate is formed at a yield greater than 70%, e.g., greater than 75%, greater than 80%, greater than 85%, greater than 90%. In terms of upper limits, the nitrile intermediate may be formed at a yield less than 100%, e.g., less than 99.9%, less than 99.5%, less than 99%, or less than 98%.

In terms of the composition of the reaction product (e.g., the solution formed after the second reaction step but before chilling to produce crystals), the content of the nitrile intermediate is also relatively high. In some embodiments, the product comprises the nitrile intermediate in an amount greater than 80 wt. %, e.g., greater than 85 wt. %, greater than 90 wt. %, or greater than 95 wt. %.

The reaction product may further comprise small amounts of unreacted reaction intermediate, e.g., as an impurity and/or side product. In some embodiments, for example, the reaction product comprises reaction intermediate in an amount less than 5 wt. %, e.g., less than 3 wt. %, less than 2 wt. %, less than 1 wt. %, or less than 0.5 wt. %.

### Further Reaction

As discussed above, the present disclosure also provides reaction pathways that include preparing the glycine derivative, e.g., alanine-N,N-diacetic acid, from the nitrile intermediate formed by the processes described herein, e.g., alanine-N,N-dinitrile. The structure of the glycine derivative is not particularly limited. As its name suggests, the glycine derivative may be a structural derivative of the amino acid glycine. In particular, the glycine derivative may be any organic compound having at least one carboxyl functional group and at least one amino functional group, wherein the carboxyl functional group and the amino functional group are separated by one carbon atom. In some embodiments, the carbon atom separating carboxyl and amino functional groups may be modified with additional moieties. In some embodiments, the nitrogen of the amino functional group may be modified with additional moieties.

In some embodiments, the glycine derivative is an organic compound having two carboxyl-containing functional groups as moieties on the nitrogen atom of the amino functional group. For example, the glycine derivative may have a chemical structure:

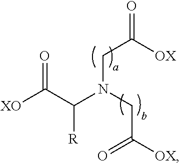

wherein a is from 0 to 5, b is from 0 to 5, and R is (C₁-C₁₀)alkyl, (C₁-C₁₀)haloalkyl, (C₁-C₁₀)alkenyl, or (C₁-C₁₀)alkyl carboxylate. In particular, a and b may correspond to their respective values in the dinitrile compound, and R may correspond to its respective value in the aldehyde. In the above chemical structure, X is hydrogen, an alkali metal, an alkaline earth metal, or ammonium. Exemplary nitrile intermediates include alanine-N,N-diacetic acid, alanine-N,N-dipropionic acid, alanine-N,N-dibutyric acid, alanine-N-acetic acid-N-propionic acid, alanine-N-acetic acid-N-butyric acid, ethyl glycine-N,N-diacetic acid, ethyl glycine-N,N-dipropionic acid, ethyl glycine-N,N-dibutyric acid, ethyl glycine-N-acetic acid-N-propionic acid, ethyl glycine-N-acetic acid-N-butyric acid, propyl glycine-N,N-diacetic acid, propyl glycine-N,N-dipropionic acid, propyl glycine-N,N-dibutyric acid, propyl glycine-N-acetic acid-N-propionic acid, and propyl glycine-N-acetic acid-N-butyric acid.

In the processes described herein, the glycine derivative may be formed by converting the nitrile functional groups of the nitrile intermediate to carboxyl functional groups. In particular, the glycine derivative may be formed by hydrolyzing the nitrile intermediate.

Hydrolysis of the nitrile intermediate is not particularly limited and any known method may be used. In some embodiments, the hydrolysis is carried out in an aqueous solution using a strong acid. In some embodiments, the hydrolysis is carried out in an aqueous solution using a strong base. Suitable strong bases include inorganic bases, such as ammonium hydroxide, calcium hydroxide, lithium hydroxide, magnesium hydroxide, potassium hydroxide, sodium hydroxide, and combinations thereof.

The hydrolysis produces the glycine derivative at high yield. In some embodiments, the glycine derivative is formed at a yield greater than 60%, e.g., greater than 65%, greater than 70%, greater than 85%, greater than 90%. In terms of upper limits, the glycine derivative may be formed at a yield less than 100%, e.g., less than 99%, less than 98%, or less than 95%.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims or the equivalents thereof.

### Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a process for preparing a nitrile intermediate, the process comprising: a first reaction step of reacting a tetra-amino compound with a hydrogen cyanide, preferably at first temperature followed by a second temperature, to form a reaction intermediate; and a second reaction step of reacting the reaction intermediate with the hydrogen cyanide and an aldehyde of the formula R—CHO, where R is (C₁-C₁₀)alkyl, (C₁-C₁₀)haloalkyl, (C₁-C₁₀)alkenyl, or (C₁-C₁₀)alkyl carboxylate, preferably at a third temperature, in an aqueous solution to form the nitrile intermediate.

Embodiment 2 is the process of embodiment(s) 1, wherein the nitrile intermediate is formed at a yield greater than 75%.

Embodiment 3 is the process of any of the preceding embodiment(s), wherein the tetra-amino compound has a formula:

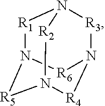

wherein R₁, R₂, R₃, R₄, R₅, and R₆ are independently (C₁-C₅)alkyl or (C₁-C₅)alkenyl, preferably (C₁-C₃)alkyl or (C₂-C₅)alkenyl.

Embodiment 4 is the process of any of the preceding embodiment(s), wherein the nitrile intermediate is alanine-N,N-dinitrile.

Embodiment 5 is the process of any of the preceding embodiment(s), wherein the reacting of the first reaction step comprises: providing a tetra-amino compound solution comprising the tetra-amino compound adjusting the pH of the tetra-amino compound solution to a pH ranging from 3.0 to 7.0; adding the hydrogen cyanide to the tetra-amino compound solution to form a first intermediate solution; heating and/or chilling the first intermediate solution to the first temperature; maintaining the first intermediate solution at the first temperature for up to 60 minutes; heating and/or chilling the heated first intermediate solution to the second temperature; and maintaining the first intermediate solution at the second temperature for up to 60 minutes.

Embodiment 6 is the process according to any of the preceding embodiment(s), wherein the reacting of the second reaction step comprises: heating and/or chilling the first intermediate solution to the third temperature; adjusting the pH of the first intermediate solution to a pH ranging from 1.5 to 7.0; adding the hydrogen cyanide and the aldehyde to the first intermediate solution at the second temperature to form a second intermediate solution; and maintaining the second intermediate solution at the third temperature for from 15 to 250 minutes to form the nitrile intermediate.

Embodiment 7 is the process according to any of the preceding embodiment(s), wherein the first reaction step and the second reaction step are carried out in the same vessel or a single vessel.

Embodiment 8 is the process according to any of the preceding embodiment(s), wherein the second reaction step comprises adding a nitrile intermediate seed to the reaction mixture.

Embodiment 9 is the process according to embodiment 8, wherein the amount of nitrile intermediate seed added is less than 1% the theoretical yield of the nitrile intermediate.

Embodiment 10 is the process of any of the preceding embodiment(s), wherein the pH of the reaction mixture is reduced by at least 2.0, optionally be adding sulfuric acid.

Embodiment 11 is the process according to any of the preceding embodiment(s), wherein the first reaction step is carried out at a pH from 3.0 to 7.0.

Embodiment 12 is the process according to any of the preceding embodiment(s), wherein the second reaction step is carried out at a pH less than 5.0.

Embodiment 13 is the process according to any of the preceding embodiment(s), wherein the first temperature is from 35° C. to 75° C.; and/or wherein the second temperature is from 50° C. to 100° C.

Embodiment 14 is the process according to any of the preceding embodiment(s), wherein the second temperature is greater than the first temperature.

Embodiment 15 is the process according to any of the preceding embodiment(s), wherein the third temperature is from 35° C. to 75° C.

Embodiment 16 is the process according to any of the preceding embodiment(s), wherein the tetra-amino compound is 1,3,5,7-tetraazaadamantane.

Embodiment 17 is the process according to any of the preceding embodiment(s), wherein R is (C₁-C₅)alkyl, and wherein R₁, R₂, R₃, R₄, R₅, and R₆ are independently (C₁-C₃)alkyl.

Embodiment 18 is the process of any of the preceding embodiment(s), further comprising forming a glycine-N,N-diacetic acid derivative.

Embodiment 19 is the process of embodiment(s) 18, wherein the glycine-N,N-diacetic acid derivative has a formula:

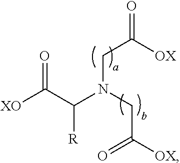

wherein: R is (C₁-C₁₀)alkyl, (C₁-C₁₀)haloalkyl, (C₁-C₁₀)alkenyl, or (C₁-C₁₀)alkyl carboxylate X is hydrogen, an alkali metal, an alkaline earth metal, or ammonium, a is from 0 to 5, and b is from 0 to 5; from the nitrile intermediate.

Embodiment 20 is the process of embodiment(s) 18 or 19, wherein the forming the glycine-N,N-diacetic acid derivative comprises hydrolyzing the nitrile intermediate.

Embodiment 21 is the process of embodiment(s) 20, wherein the hydrolyzing comprises reacting the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof.

Embodiment 22 is the process of any one of embodiment(s) 18-21, wherein the glycine-N,N-diacetic acid derivative is alanine-N,N-diacetic acid derivative.

Embodiment 23 is the process of any one of embodiment(s) 18-22, wherein the glycine-N,N-diacetic acid derivative is formed at a yield of at least 60%.

### EXAMPLES

The present disclosure will be further understood by reference to the following examples.

Tetraazaadamantane (6.55 g) was added to 50 mL of deionized water at room temperature to produce a tetra-amino compound solution. Sulfuric acid was added to the tetra-amino compound solution so as to adjust the pH to 5.0. Hydrogen cyanide (24 ml; about 15 g) was then added to the tetra-amino compound solution over 30 minutes to form a first intermediate solution. The pH of the first intermediate solution was maintained at 5.0 by addition of sulfuric acid, as needed. While the hydrogen cyanide was being added, the first intermediate solution was heated to a first temperature of 50° C. The first intermediate solution was maintained at the first temperature for about 15 minutes, and then was headed to the second temperature of 70° C. The first intermediate solution was maintained at the second temperature for four minutes.

The first intermediate solution was allowed to cool down to 50° C. The first intermediate solution was then seeded with crystalline alanine-N,N-diacetonitrile (0.013 g). The pH of the first intermediate solution was adjusted to about 3.0-3.5 by the addition of sulfuric acid. A second portion of hydrogen cyanide (10 ml; about 5 g) and acetaldehyde (7.8 g) were added to the first intermediate solution to form a second intermediate solution. After the addition of the hydrogen cyanide and the acetaldehyde, the second intermediate solution was stirred at 50° C. for 180 minutes.

After 180 minutes, the second intermediate solution was cooled to 5° C. While the solution cooled, a crystalline reaction product formed. The solid crystals were filtered and dried overnight. A sample of the reaction product was analyzed and revealed that the reaction product comprised about 97.05 wt. % crystalline alanine-N,N-dinitrile (nitrile intermediate) and 0.20 wt. % ((cyanomethyl)amino)acetonitrile (reaction intermediate). Conversion was greater than 99%, and yield was 97%, both of which were significantly higher than expected.

We claim:

1. A process for preparing a nitrile intermediate, the process comprising:
a first reaction step of reacting a tetra-amino compound with a hydrogen cyanide to form a reaction intermediate; and
a second reaction step of reacting the reaction intermediate with the hydrogen cyanide and an aldehyde of the formula R—CHO, where R is (C₁-C₁₀)alkyl, (C₁-C₁₀)haloalkyl, (C₁-C₁₀)alkenyl, or (C₁-C₁₀)alkyl carboxylate, in an aqueous solution to form the nitrile intermediate.

2. The process of claim 1, wherein the nitrile intermediate is formed at a yield greater than 75%.

3. The process of claim 1, wherein the reacting of the first reaction step comprises:
providing a tetra-amino compound solution comprising the tetra-amino compound adjusting the pH of the tetra-amino compound solution to a pH ranging from 3.0 to 7.0;
adding the hydrogen cyanide to the tetra-amino compound solution to form a first intermediate solution;
heating and/or chilling the first intermediate solution to a first temperature;
maintaining the first intermediate solution at the first temperature for up to 60 minutes;
heating and/or chilling the heated first intermediate solution to a second temperature; and
maintaining the first intermediate solution at the second temperature for up to 60 minutes.

4. The process of claim 3, wherein the first temperature is from 35° C. to 75° C.; and/or wherein the second temperature is from 50° C. to 100° C.

5. The process of claim 3, wherein the second temperature is greater than the first temperature.

6. The process of claim 1, wherein the reacting of the second reaction step comprises:
heating and/or chilling the first intermediate solution to a third temperature;
adjusting the pH of the first intermediate solution to a pH ranging from 1.5 to 7.0;
adding the hydrogen cyanide and the aldehyde to the first intermediate solution at the second temperature to form a second intermediate solution; and
maintaining the second intermediate solution at the third temperature for from 15 to 250 minutes to form the nitrile intermediate.

7. The process of claim 6, wherein the third temperature is from 35° C. to 75° C.

8. The process of claim 1, wherein the first reaction step and the second reaction step are carried out in one vessel.

9. The process of claim 1, wherein the second reaction step comprises adding a nitrile intermediate seed to the reaction mixture.

10. The process of claim 9, wherein the amount of nitrile intermediate seed added is less than 1% the theoretical yield of the nitrile intermediate.

11. The process of claim 1, wherein the tetra-amino compound has a formula: 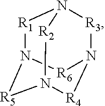
wherein R₁, R₂, R₃, R₄, R₅ and R₆ are independently (C₁-C₅)alkyl or (C₁-C₅)alkenyl.

12. The process of claim 1, wherein the nitrile intermediate is alanine-N,N-dinitrile.

13. The process of claim 1, wherein the first reaction step is carried out at a pH from 3.0 to 7.0.

14. The process of claim 1, wherein the second reaction step is carried out at a pH less than 5.0.

15. The process of claim 1, wherein the tetra-amino compound is 1,3,5,7-tetraazaadamantane.

16. The process of claim 1, further comprising forming a glycine-N,N-diacetic acid derivative.

17. The process of claim 16, wherein the glycine-N,N-diacetic acid derivative has a formula 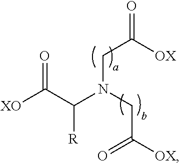
wherein:
R is (C₁-C₁₀)alkyl, (C₁-C₁₀)haloalkyl, (C₁-C₁₀)alkenyl, or (C₁-C₁₀)alkyl carboxylate
X is hydrogen, an alkali metal, an alkaline earth metal, or ammonium,
a is from 0 to 5, and
b is from 0 to 5.

18. The process of claim 16, wherein the forming the glycine-N,N-diacetic acid derivative comprises hydrolyzing the nitrile intermediate with an inorganic hydroxide selected from the group consisting of ammonium hydroxide, calcium hydroxide, lithium hydroxide, magnesium hydroxide, potassium hydroxide, sodium hydroxide, and combinations thereof.

19. The process of claim 16, wherein the glycine-N,N-diacetic acid derivative is alanine-N,N-diacetic acid derivative.

20. The process of claim 16, wherein the glycine-N,N-diacetic acid derivative is formed at a yield of at least 60%.
